(19) United States
(12) Patent Application Publication
(10) Pub. No.: US 2020/0324137 A1
(43) Pub. Date: Oct. 15, 2020

(54) ULTRAVIOLET IRRADIATION DEVICE, ATTACHMENT AND ELASTIC MEMBER FOR USE IN ULTRAVIOLET IRRADIATION DEVICE, AND ULTRAVIOLET IRRADIATION METHOD

(71) Applicants: PUBLIC UNIVERSITY CORPORATION NAGOYA CITY UNIVERSITY, Aichi (JP); Ushio Denki Kabushiki Kaisha, Tokyo (JP); ISHIZUKA GLASS CO., LTD., Aichi (JP)

(72) Inventors: Akimichi MORITA, Aichi (JP); Hideyuki MASUDA, Tokyo (JP); Makoto KIMURA, Tokyo (JP); Yuji OGAWA, Iwakura-shi (JP); Miki YOSHIDA, Iwakura-shi (JP)

(73) Assignees: PUBLIC UNIVERSITY CORPORATION NAGOYA CITY UNIVERSITY, Aichi (JP); Ushio Denki Kabushiki Kaisha, Tokyo (JP); ISHIZUKA GLASS CO., LTD., Aichi (JP)

(21) Appl. No.: 16/757,957

(22) PCT Filed: Dec. 20, 2017
(86) PCT No.: PCT/JP2017/045800

§ 371 Date: Apr. 21, 2020

(51) Int. Cl. A61N 5/06

(52) U.S. Cl. CPC A61N 5/0616; A61N 2005/0661; A61N 2005/065; A61N 2005/0632

(57) ABSTRACT

Provided is an ultraviolet irradiation device capable of increasing the irradiance at an affected site (target cells) even when ultraviolet light having the same intensity is emitted. This ultraviolet irradiation device is provided with: a device body configured to be capable of emitting ultraviolet light from a light emission unit; an ultraviolet-transparent substrate disposed on the light emission unit; and an elastic member which is disposed on the surface of the substrate facing away from the device body and made of an ultraviolet-transparent material.

### TECHNICAL FIELD

The present invention relates to an ultraviolet irradiation device, an attachment and an elastic member for use in an ultraviolet irradiation device, and an ultraviolet irradiation method.

### BACKGROUND ART

Phototherapy includes infrared treatment using near-infrared light and ultraviolet treatment using light such as UVA (wavelength: 320 nm to 400 nm) and UVB (wavelength: 290 nm to 320 nm). In recent years, ultraviolet treatment has become widespread particularly as a treatment for skin diseases such as vitiligo, psoriasis, and atopic dermatitis. For example, Patent Document 1 listed below discloses a therapeutic device using an excimer lamp.

Mechanisms of action of these treatment techniques include (1) effects on humoral factors such as cytokines and chemokines, (2) changes in expression of cell surface molecules such as adhesion molecules, (3) induction of apoptosis of pathogenic cells, and (4) induction of regulatory T cells. Among these, the mechanism (3) is particularly important. In diseases such as psoriasis, atopic dermatitis, and T-cell lymphoma, in which pathogenic T cells infiltrate the dermis as a disease state, it has been shown that the T cells are irradiated with ultraviolet light, whereby the infiltrating T cells are fallen under a state of apoptosis and removed, so that the lesion is improved.

### PRIOR ART DOCUMENT

### Patent Document

Patent Document 1: JP-B-4670780

### Non-Patent Documents

Non-Patent Document 1: Anderson R P, Parrish J A et al., Optics of the skin. Clinical photomedicine (Lim H W, Soter N A, Ed), Marcel Dekker, New York, 1993, 19-35

Non-Patent Document 2: Mark Allen Evereit et al., Penetration of epidermis by ultraviolet rays., Photochem Photobiol. 1966 July; 5 (7): 533-42.

Non-Patent Document 3: Takeshi Horio, “Photodermatology V. Basic Knowledge of Ultraviolet Light Required for Phototherapy”, Skin Science, Vol. 15, No, 1, Feb. 2016

### SUMMARY OF THE INVENTION

### Problems to be Solved by the Invention

In ultraviolet therapy, ultraviolet light is directly applied to the skin in an affected area. While ultraviolet light arrives from an outer surface of the skin to a layer in which a T cell of a target cell is infiltrated, the ultraviolet light is absorbed by light-absorbing substances such as water molecules, melanin, and hemoglobin or diffused by cells forming the stratum corneum, epidermis, and dermis. As a result, irradiance of ultraviolet light attenuates as a traveling distance increases.

For example, when UVB light is applied from the outer surface of the skin, an amount of ultraviolet light reaching the dermis is about 10% (see Non-Patent Documents 1 to 3 listed above).

That is, when ultraviolet light is applied from the outer surface of the skin, there is a possibility that ultraviolet light with sufficient irradiance has not reached the affected area (target cell). On the other hand, there is a limit to increasing intensity of ultraviolet light itself because it may adversely affect healthy cells. Under such circumstances, according to conventional ultraviolet treatment methods, the treatment period may be prolonged.

In view of such circumstances, an object of the present invention is to provide an ultraviolet irradiation device that can improve irradiance applied to an affected area (target cell) even when ultraviolet light of the same intensity is emitted. Another object of the present invention is to provide an attachment and an elastic member for use in such an ultraviolet irradiation device. Another object of the present invention is to provide an ultraviolet irradiation method.

### Means for Solving the Problems

An ultraviolet irradiation device according to the present invention includes a device body configured to be able to emit ultraviolet light from a light emitting portion,

a substrate disposed at the light emitting portion and exhibiting transparency to ultraviolet light, and

an elastic member placed on a surface of the substrate opposite to the device body and formed of a material having transparency to the ultraviolet light.

Hemoglobin which is one of the light-absorbing substances is contained in blood and circulates in blood vessels in the skin and capillaries with time. According to the above configuration, in a state where the elastic member is in contact with the outer surface of the skin, the ultraviolet light is applied while the elastic member is pressed through the substrate, whereby ultraviolet irradiation is possible while inflow of blood to an affected area is temporarily blocked.

That is, the elastic member has transparency to the ultraviolet light and elasticity. Since the elastic member has elasticity, when the elastic member is pressed, the shape of the elastic member is easily deformed according to a curved surface formed by the outer surface of the skin. Consequently, the outer surface of the skin and the elastic member easily come into surface contact. Since the elastic member has transparency with respect to the ultraviolet light, the ultraviolet light emitted from the device body is transmitted through the elastic member and guided to the inside of the skin.

Consequently, absorption of the ultraviolet light by the light-absorbing substance contained in the affected area is reduced. In addition, the epidermis and the dermis are compressed, so that a distance from the outer surface of the skin to a target cell can be shortened, and therefore, attenuation of irradiance is additionally suppressed. As a result, even when ultraviolet light having the same intensity is emitted, the irradiance to the affected area is improved as compared with conventional devices. The ultraviolet irradiation device according to the present invention can be used as an ultraviolet treatment device.

The elastic member has a transmittance for ultraviolet light of preferably 90% or more, and more preferably 93% or more, when a thickness including surface reflection is 1 mm. It is preferable that when the elastic member having a thickness of 1 to 10 mm is bent by hand, the elastic member has such an elasticity that it can be bent without cracking. For example, a Young's modulus is preferably 3 MPa or less, and more preferably 1 MPa or less.

The elastic member is formed of, for example, an organic-inorganic hybrid composition (X), and the organic-inorganic hybrid composition (X) can be realized by having no phenyl group in its molecule, having only a methyl group in its side chain, and having a skeleton composed of dimethylpolysiloxane having a hydroxy terminal. According to the organic-inorganic hybrid composition having no phenyl group in its molecule, having only a methyl group in its side chain, and having a skeleton formed of dimethylpolysiloxane having a hydroxy terminal, an ultraviolet transmittance is excellent, and such a characteristic that elasticity (high flexibility) is provided is realized.

The organic-inorganic hybrid composition (X) is preferably a product formed by dehydration-condensation of the dimethylpolysiloxane (A), aluminum alkoxide (B), and silicon alkoxide (C).

The device body preferably has a size and weight that allow the device body to be gripped by hand. With this configuration, it is possible to apply ultraviolet light while pressing the outer surface of the skin together with the device body in a state where the device body is gripped by hand. Consequently, the irradiance to the affected area can be improved by simple processing as compared with the conventional devices.

The device body may include an ultraviolet light source, or may have a configuration in which ultraviolet light is guided from an ultraviolet light source provided in another place via a light guide member such as an optical fiber.

The ultraviolet irradiation device may have an attachment formed of a frame-shaped member including an opening region and detachably attached to the device body. The elastic member may be fitted into the opening region, and an outer peripheral portion of the elastic member may be fixed to the device body via the attachment.

Since the elastic member is in contact with the outer surface of the skin, it is assumed that the elastic member is used up in consideration of hygiene. Thus, when radiotherapy is applied to many patients, it is conceivable to attach and detach the elastic member to and from the device body each time. According to the above configuration, since the elastic member can be easily attached to the device body, preparation for ultraviolet irradiation is simplified.

The elastic member has a first surface located on a side closer to the substrate and a second surface opposite to the first surface, and the second surface can be disposed projecting opposite to the device body relative to the attachment.

According to this configuration, since the elastic member projects from the device body at an opposite side thereof, the elastic member can be easily brought into contact with the outer surface of the skin by pressing the device body toward the skin outer surface side.

The elastic member may have a step portion formed at a position between the first surface and the second surface. When the frame-shaped member of the attachment comes into contact with the step portion, the elastic member can be fitted into the opening region.

According to this configuration, the elastic member can be easily attached to the device body with part of surfaces (the second surface) projecting opposite to the device body. In the elastic member, a first portion including a first surface which is a surface on a side closer to a substrate and a second portion including a second surface which is a surface opposite to the substrate are continuous in a direction perpendicular to the first surface and the second surface, and an area of the first portion (area of the first surface) is larger than a second portion (area of the second surface), so that the step portion may be formed.

The elastic member may have a thickness of 3 mm to 10 mm. Although the elastic member has transparency to ultraviolet light, a member having a transmittance of 100% is practically difficult. Thus, part of incident light is inevitably diffused and absorbed. If the thickness of the elastic member exceeds 10 mm, the amount of diffusion and absorption of ultraviolet light in the elastic member increases, so that an effect of improving the irradiance to an affected area decreases. On the other hand, when the thickness of the elastic member is less than 3 mm, the elastic member is less likely to come into surface contact along a curvature of the outer surface of the skin, so that an original effect of temporarily blocking a blood flow is weakened.

An ultraviolet irradiation method according to the present invention includes placing an elastic member, formed of a material having transparency to ultraviolet light, on a surface of an irradiation target region, and in a state where in a surface of the elastic member, which is opposite to the irradiation target region, a substrate exhibiting transparency to ultraviolet light is in contact with the elastic member, applying the ultraviolet light via the substrate and the elastic member.

### Effect of the Invention

The present invention can realize the ultraviolet irradiation device which can improve the irradiance applied to an affected area.

### BRIEF DESCRIPTION OF THE DRAWINGS

FIG. 1 is a perspective view schematically showing a configuration of one embodiment of an ultraviolet irradiation device.

FIG. 2 is a schematic top view of the ultraviolet irradiation device shown in FIG. 1.

FIG. 3 is a schematic front view of the ultraviolet irradiation device shown in FIG. 1.

FIG. 4 is a view showing the ultraviolet irradiation device shown in FIG. 1 in a state where some parts are disassembled.

FIG. 5A is a schematic perspective view showing an elastic member and an attachment.

FIG. 5B is a schematic front view of a structure shown in FIG. 5A.

FIG. 6 is a perspective view schematically showing a structure of the attachment.

FIG. 7A is a perspective view schematically showing a structure of the elastic member.

FIG. 7B is a schematic front view of the structure shown in FIG. 7A.

FIG. 8 is a schematic cross-sectional view obtained by cutting the ultraviolet irradiation device with a line A1-A1 in FIG. 3.

FIG. 9 is a view schematically showing a usage mode of the ultraviolet irradiation device.

FIG. 10 is a schematic view in which a contact region between the elastic member and a skin outer surface is enlarged.

FIG. 11A is a result obtained by measuring a color difference by an SCI method when the elastic member is pressed against the skin of a subject A.

FIG. 11B is a result obtained by measuring the color difference by the SCI method when the elastic member is pressed against the skin of a subject B.

FIG. 11C is a result obtained by measuring the color difference by the SCI method when the elastic member is pressed against the skin of a subject C.

FIG. 11D is a graph comparing results obtained by calculating a difference value a between an L value and an a value for each of the subjects A, B, and C based on the results obtained in FIGS. 11A to 11C.

FIG. 12A is a graph showing results obtained by measuring a transmittance spectrum with respect to light in a state where a thickness t of the elastic member is changed to 1 mm, 3 mm, 5 mm, and 10 mm.

FIG. 12B is a graph obtained by enlarging a partial region of FIG. 12A.

FIG. 13 is two photographs of a surface of the skin of the same subject when a case where the subject is irradiated with ultraviolet light via the elastic member and a case where the subject is irradiated with the ultraviolet light without the elastic member are compared.

FIG. 14 is a perspective view schematically showing a configuration of another embodiment of the ultraviolet irradiation device.

FIG. 15 is a perspective view schematically showing a configuration of another embodiment of the ultraviolet irradiation device.

### MODE FOR CARRYING OUT THE INVENTION

An embodiment of an ultraviolet irradiation device according to the present invention will be described with reference to the drawings. The following drawings are schematically illustrated, and the dimensional ratio in the drawings does not necessarily coincide with the actual dimension ratio. Also, the dimensional ratios in the drawings do not necessarily coincide with the explanation.

### [Device Structure]

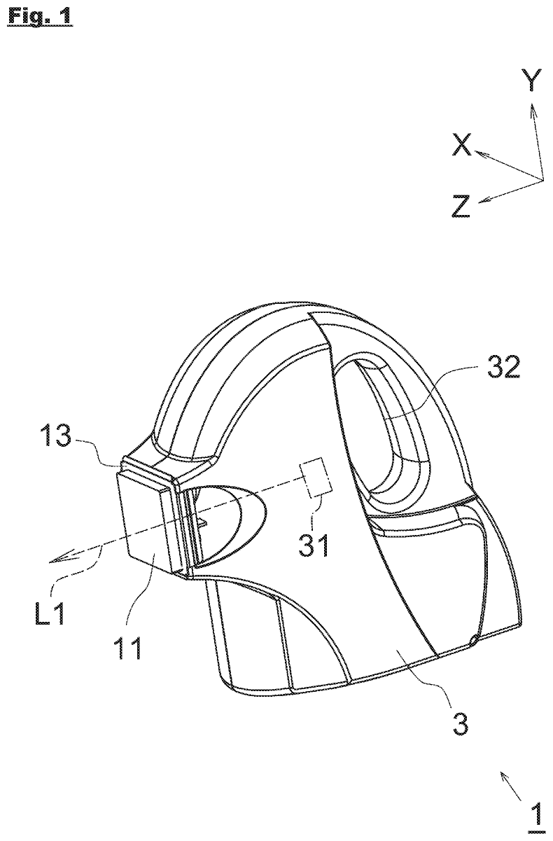

FIGS. 1 to 8 are views schematically showing an ultraviolet irradiation device according to the present embodiment or parts of the device. FIG. 1 is a schematic perspective view of an ultraviolet irradiation device 1. Hereinafter, description will be made with reference to an XYZ coordinate system shown in FIG. 1 as appropriate.

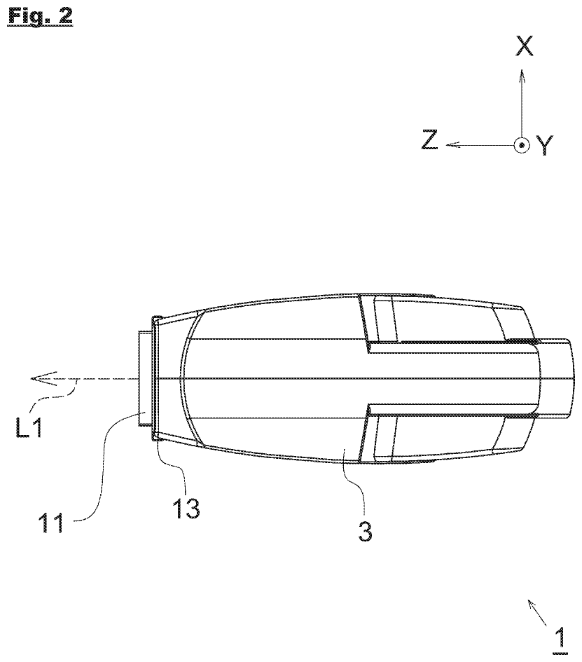

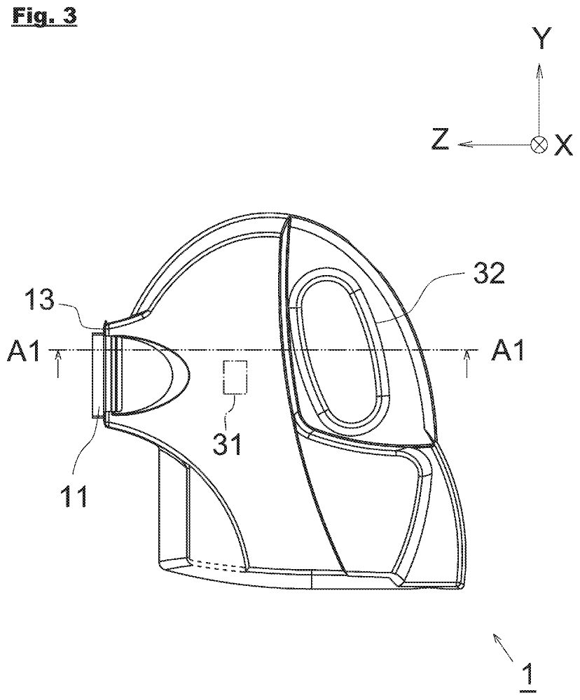

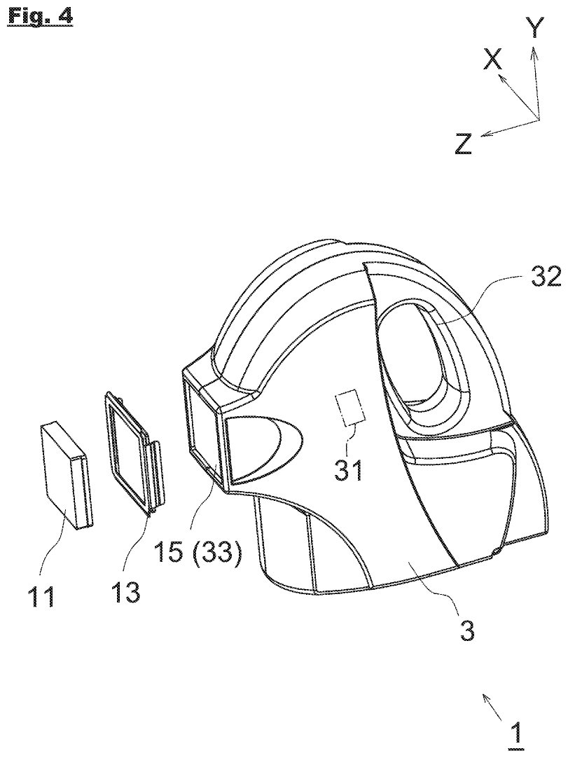

FIG. 2 is a schematic top view of the ultraviolet irradiation device 1, and corresponds to the drawing when the ultraviolet irradiation device 1 is viewed along a Y direction. FIG. 3 is a schematic front view of the ultraviolet irradiation device 1, and corresponds to the drawing when the ultraviolet irradiation device 1 is viewed along an X direction. FIG. 4 is a view showing the ultraviolet irradiation device 1 shown in FIG. 1 in a state where some parts are disassembled.

The ultraviolet irradiation device 1 includes a device body 3, an elastic member 11, an attachment 13, and a substrate 15 (see FIG. 4). As shown in FIG. 4, in the present embodiment, the substrate 15 is attached to the device body 3. Furthermore, the ultraviolet irradiation device 1 of the present embodiment includes a light source 31 that emits ultraviolet light L1 into the device body 3, and a gripping portion 32 for gripping the device body 3 itself. FIGS. 1 to 3 do not show the substrate 15 for convenience of illustration.

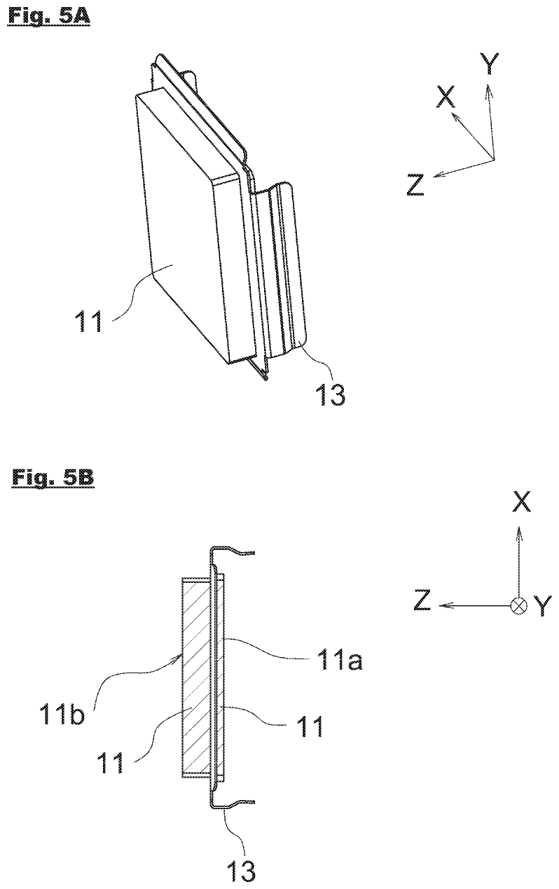

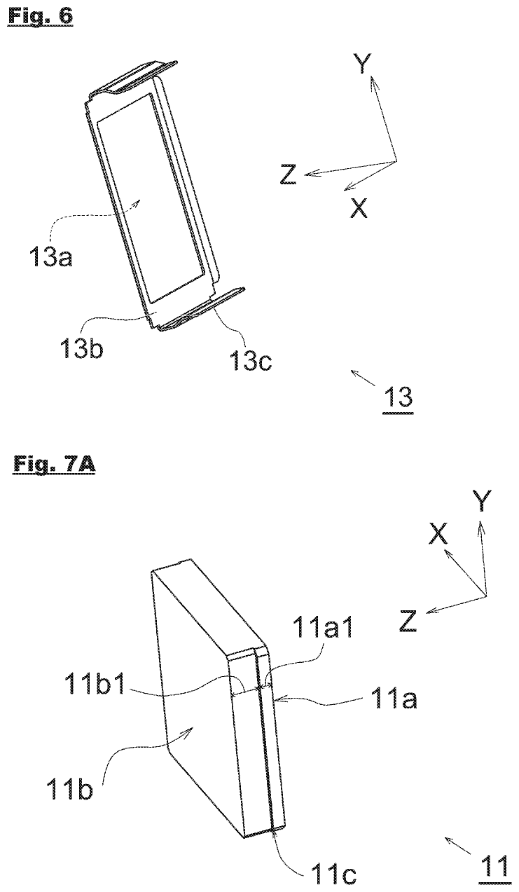

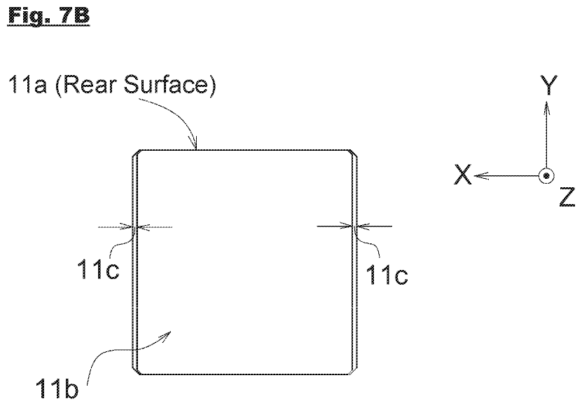

FIG. 5A is a schematic perspective view showing the elastic member 11 and the attachment 13. FIG. 5B corresponds to a front view of FIG. 5A (the drawing when FIG. 5A is viewed along the Y direction). FIG. 6 is a perspective view schematically showing a structure of the attachment 13. FIG. 7A is a perspective view schematically showing a structure of the elastic member 11. FIG. 7B is a front view schematically showing the structure of the elastic member 11.

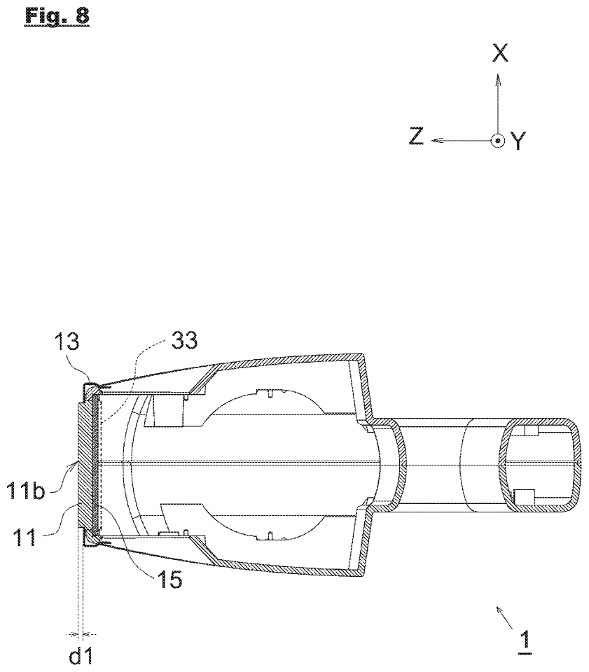

FIG. 8 is a schematic cross-sectional view obtained by cutting the ultraviolet irradiation device 1 with a line A1-A1 in FIG. 3.

As shown in FIG. 4, the ultraviolet irradiation device 1 includes a region (light emitting portion 33) for emitting the ultraviolet light L1 in the device body 3. The light emitting portion 33 constitutes a window for guiding the ultraviolet light L1 to the outside of the device body 3. In the configuration shown in FIG. 4, a substrate 15 having transparency to the ultraviolet light L1 is fitted into the light emitting portion 33 (window), and the ultraviolet light L1 is guided to the outside of the device body 3 via the substrate 15.

In the present embodiment, the elastic member 11 is placed on a surface of the substrate 15 in a +Z direction. The surface of the substrate 15 in the +Z direction refers to a surface of the substrate 15 in a light emitting direction (a surface opposite to the device body 3). The elastic member 11 is continuous so as not to drop off from the device body 3 by the attachment 13. By fixing by the attachment 13, a surface of the elastic member 11 comes into contact with a surface of the substrate 15. FIGS. 5A and 5B show that the surface of the elastic member 11 and the surface of the substrate 15 are in contact with each other. Another member having ultraviolet transmittance may be interposed on the surface of the substrate 15 on the light source 31 side. In this case, the ultraviolet light L1 is guided to the outside of the device body 3 via the light emitting portion 33 constituting the window, the another member, and the substrate 15.

As shown in FIG. 6, the attachment 13 is constituted by a frame-shaped member 13b having an opening region 13a formed inside, and a pair of opposing sides of an outer peripheral portion are provided with claw portions 13c. The claw portion 13c constitutes, for example, a leaf spring, and is engaged with a receiving portion (not shown) provided in the device body 3, so that the attachment 13 and the device body 3 are fixedly connected. The attachment 13 can be easily removed from the device body 3 by operating the claw portion 13c.

As shown in FIGS. 7A and 7B, the elastic member 11 has two facing surfaces (first surface 11a and second surface 11b) parallel to an XY plane and a step portion 11c provided at a position between the two surfaces. More specifically, the elastic member 11 is located on a side closer to the substrate 15 and has a first portion 11a1 having the first surface 11a with a large area and a second portion 11b1 located on the opposite side (light emission side) of the substrate 15 and having a second surface 11b with a smaller area than the first surface 11a, and these portions are formed continuously.

The length of each side of the elastic member 11 on the XY plane in the second portion 11b1 is shorter than the length of each side on the XY plane that forms the outer peripheral portion of the opening region 13a of the attachment 13. On the other hand, the length of each side of the elastic member 11 on the XY plane in the first portion 11a1 is longer than the length of each side on the XY plane that forms the outer peripheral portion of the opening region 13a of the attachment 13. A thickness of the elastic member 11 (length in a Z direction) is larger than a thickness of a portion of the attachment 13 that constitutes the frame-shaped member 13b.

When configured in this manner, the second portion 11b1 of the elastic member 11 can pass through the opening region 13a of the attachment 13, and on the other hand, the first portion 11a1 of the elastic member 11 cannot pass through the opening region 13a of the attachment 13. That is, when the elastic member 11 is fitted into the opening region 13a of the attachment 13, the elastic member 11 is fixed at the step portion 11c located at a boundary between the first portion 11a1 and the second portion 11b1 in a state of being in contact with the outer peripheral portion of the opening region 13a of the attachment 13. At this time, on both sides (±Z direction) of the opening region 13a, a portion of the elastic member 11, that is, the first surface 11a and the second surface 11b project outside the opening region 13a.

When the elastic member 11 is attached to the device body 3 in a stale of being fitted into the opening region 13a of the attachment 13, as shown in FIG. 8, the second surface 11b of the elastic member 11 projects by a length d1 on the opposite side (+Z direction) of the device body 3 relative to the attachment 13.

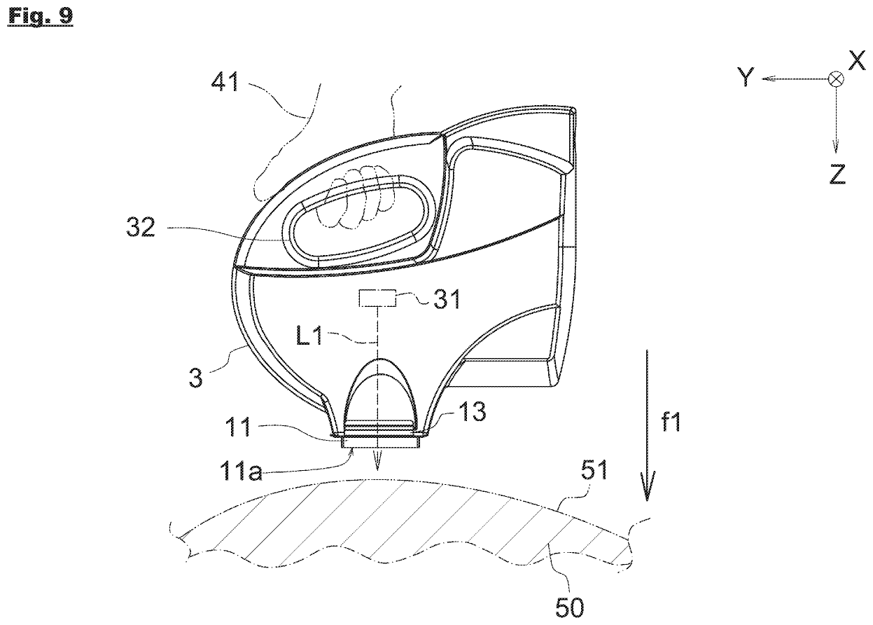

FIG. 9 is a view schematically showing a usage mode of the ultraviolet irradiation device 1 according to the present embodiment. The elastic member 11 is opposed to an irradiation subject (patient) 50 with an operator 41 gripping a gripping portion 32. In this state, the operator 41 brings the elastic member 11 into contact with a skin outer surface 51, which is an irradiation target region of the irradiation subject 50, and further applies a load f1 from the device body 3 toward the skin outer surface 51.

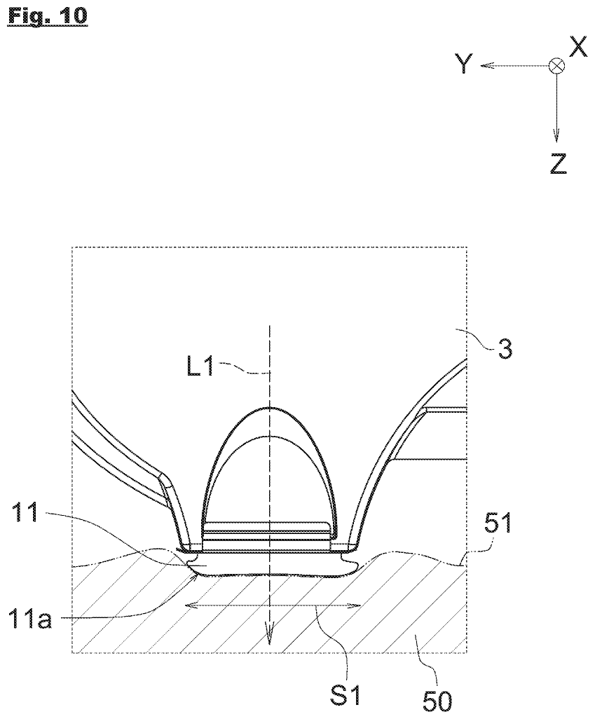

As will be described later, the elastic member 11 is formed of a material having high transparency to ultraviolet light and elasticity. Thus, the shape of the elastic member 11 changes along a curved surface of the skin outer surface 51, and the elastic member 11 comes into surface contact with the skin outer surface 51. The skin in the region is compressed inward by applying the load f1 to the skin outer surface 51. FIG. 10 is a view schematically showing this state.

As shown in FIG. 10, in a region S1 where a load is applied, the skin outer surface 51 is compressed inside the body. The surface (first surface 11a) of the elastic member 11 is deformed along the curved surface of the skin outer surface 51.

In such a state, the ultraviolet irradiation device 1 irradiates the irradiation subject (patient) 50 with the ultraviolet light L1. At this time, since the load f1 is applied to the region S1, inflow of blood is temporarily blocked or reduced. As a result, the number of hemoglobins in the region S1 temporarily decreases. Since hemoglobin is one of factors absorbed by the ultraviolet light L1, irradiance of the ultraviolet light L1 reaching an affected region existing inside the skin is increased by reducing the number of hemoglobins.

In particular, since the elastic member 11 comes into surface contact with the skin outer surface 51 in the region S1, the load f1 is applied to the irradiation subject 50 through the skin outer surface 51 via the device body 3, so that the effect of temporarily blocking the inflow of blood in the region S1 is exhibited.

### [Elastic Member 11]

As a member that transmits ultraviolet light (for example, UVB light), hard materials such as quartz glass and fluorite (calcium fluoride) have been known. However, a material that exhibits a transmittance of 90% or more to ultraviolet light and is easily bent (has elasticity) by hand at a thickness of 1 mm or more has been scarcely known. When the elastic member 11 is formed of a material described below, although this material is very soft to be deformed along a curved surface having a diameter of 200 mm with a force of 1 kgf, the material has a low stickiness, and the transmittance for UVB light is 90% or more in a 1 mm thick member.

### (Material)

The elastic member 11 included in the ultraviolet irradiation device 1 of the present embodiment is formed of an organic-inorganic hybrid composition (X). The organic-inorganic hybrid composition (X) can be realized by having no phenyl group in its molecule, having only a methyl group in its side chain, and having a skeleton composed of dimethylpolysiloxane having a hydroxy terminal. As an example, the organic-inorganic hybrid composition is preferably a product containing dimethylpolysiloxane (A), aluminum alkoxide (B), and silicon alkoxide (C) and produced by crosslinking with a dehydration condensation reaction of these materials.

The organic-inorganic hybrid composition (X) has a structure in which a polysiloxane having a siloxane bond is three-dimensionally and complexly crosslinked. Thus, the organic-inorganic hybrid composition (X) has a structure similar to that of a so-called inorganic glass, and suitable properties such as heat resistance and ultraviolet resistance can be obtained.

The dimethylpolysiloxane (A) having a hydroxy terminal is a material that forms a skeleton structure of the organic-inorganic hybrid composition (X) and is a silicon compound having no phenyl group in its molecule and having only a methyl group in its side chain. Although the transmittance of the organic-inorganic hybrid composition (X) of the type containing a phenyl group is 75% or more in a wavelength region of 300 nm or more, the phenyl group absorbs ultraviolet light in the 260 nm region, so that ultraviolet light is hardly transmitted. On the other hand, the absorption of ultraviolet light can be prevented by using as a raw material the hydroxy-terminated dimethylpolysiloxane (A) having no phenyl group.

According to the organic-inorganic hybrid composition (X) having a skeleton formed of the dimethylpolysiloxane (A) having no phenyl group in its molecule, having only a methyl group in its side chain, and having a hydroxy terminal, the organic-inorganic hybrid composition (X) is resistant to bending and is less likely to be damaged when curved, so that high elasticity is ensured.

In order to increase crosslinking reactivity between the dimethylpolysiloxanes (A) or between the dimethylpolysiloxane (A) and the alkoxide molecule (B) or (C), the terminal portion of the dimethylpolysiloxane (A) is substituted with a hydroxy group. The hydroxy-terminated dimethylpolysiloxane (A) is a molecule serving as the structural skeleton of the organic-inorganic hybrid composition (X), and is generally selected from a molecular weight (weight average molecular weight) of from 500 to 30,000.

The aluminum alkoxide (B) has a role of forming a network structure of molecules by a condensation reaction with a hydroxy group which is a terminal portion of the hydroxy-terminated dimethylpolysiloxane (A). Examples of the aluminum alkoxide (B) include various types of aluminum alkoxides including aluminum sec-butoxide, aluminum tert-butoxide, monosec-butoxyaluminum diisopropylate (also known as aluminum (2-butanolate) di (2-propanolate)) and the like. From the viewpoint of securing a high transmittance for ultraviolet light, aluminum sec-butoxide is particularly preferable.

The aluminum alkoxide (B) has higher reactivity such as hydrolysis and condensation than the silicon alkoxide (C). As a result, the aluminum alkoxide (B) causes hydrolysis without using a catalyst such as an acid or a base. Specifically, the aluminum alkoxide (B) undergoes a condensation reaction with a hydroxy group of the hydroxy-terminated dimethylpolysiloxane (A) without using a tin-based reaction accelerator such as dibutyltin diacetate, dibutyltin dilaurate, dioctyltin dilaurate, bis(acetoxydibutyltin) oxide, and bis(lauroxydibutyltin) oxide, and a crosslink can be formed.

From the viewpoint of ensuring high transparency to ultraviolet light, a high energy band gap is required for a metal oxide derived from a reaction product of a highly reactive metal alkoxide contained in the organic-inorganic hybrid composition (X). The energy band gap of Al₂O₃ is 6.9 eV, and an absorption edge is 179.7 nm. Therefore, the aluminum alkoxide (B) achieves high transparency to ultraviolet light.

The silicon alkoxide (C) also has a role of forming a network structure of molecules by a condensation reaction with a hydroxy group which is a terminal portion of the hydroxy-terminated dimethylpolysiloxane (A). Examples of the silicon alkoxide (C) include various types of silicon alkoxides including, such as tetraethoxysilane, tetramethoxysilane, tetrabutoxysilane, tetraisopropoxysilane, tetrapropoxysilane, methyltriethoxysilane, ethyltriethoxysilane, n-propyltriethoxysilane, isobutyltriethoxysilane, n-hexyltriethoxysilane, n-octyltriethoxysilane, n-dodecyltriethoxysilane, methyltrimethoxysilane, ethyltrimethoxysilane, n-propyltrimethoxysilane, n-butyltrimethoxysilane, isobutyltrimethoxysilane, n-hexyltrimethoxysilane and n-dodecyltrimethoxysilane, and condensates thereof. As a silicon alkoxide oligomer as the condensate. “KC-89S” commercially manufactured by Shin-Etsu Chemical Co., Ltd. can also be used.

The hydroxy-terminated dimethylpolysiloxane (A), the aluminum alkoxide (B), and the silicon alkoxide (C) are mixed, for example:, in an alcohol solvent. Alcohol dissolves alkoxide and is mixed with the dimethylpolysiloxane (A). After mixing each material, the alcohol solvent used is removed by evaporation by drying.

To a precursor crosslinked by dehydration condensation between the aluminum alkoxide (B) and the terminal hydroxy group of the dimethylpolysiloxane (A), the silicon alkoxide (C) is further crosslinked by dehydration condensation. Thereafter, moisture in the air is absorbed from a crosslinked product of the above three compounds, that is, the surface of the organic-inorganic hybrid composition (X). Hydrolysis of the aluminum alkoxide (B) and the silicon alkoxide (C) proceeds by the moisture absorbed from the surface of the composition. In addition, dehydration condensation with the hydroxy-terminated dimethylpolysiloxane (A) is promoted. Hydrolysis of the aluminum alkoxide (B) and the silicon alkoxide (C) is further induced due to water generated by the condensation.

As described above, the hydrolysis of the alkoxide and the dehydration condensation of the polysiloxane occur sequentially, and cross-linking and curing reactions from the surface of the organic-inorganic hybrid composition (X) proceed gradually throughout the entire interior of the organic-inorganic hybrid composition (X). Finally, the organic-inorganic hybrid composition (X) which has been a fluid is crosslinked, cured, and then molded into a predetermined shape (for example, the shape described with reference to FIGS. 7A and 7B), so that the elastic member 11 having high transparency and elasticity to ultraviolet light is formed. In the stage before molding, for example, a curing treatment may be performed by performing a heating treatment at 70° C. or more and 200° C. or less for 4 hours or more and 12 hours or less.

As the hydroxy-terminated dimethylpolysiloxane (A) used for the organic-inorganic hybrid composition (X), two or more types of hydroxy-terminated dimethylpolysiloxanes having different weight average molecular weights may be used.

### (Example Regarding Constituent Material of Elastic Member 11)

### Dimethylpolysiloxane (A): Examples 1 to 6, Comparative Examples 1 and 2

As the hydroxy-terminated dimethylpolysiloxane (A), two types having different molecular weights (average degree of polymerization) were used. As low molecular weight dimethylpolysiloxane (A1), YF3800 (weight average molecular weight: 3,500) manufactured by Momentive Performance Materials Japan Kk was used, and as high molecular weight dimethylpolysiloxane (A2), XF3905 (weight average molecular weight: 20,000) manufactured by Momentive Performance Materials Japan Kk was used.

### Aluminum Alkoxide (B): Examples 1 to 6

As the aluminum alkoxide (B), aluminum sec-butoxide (aluminum sec-butyrate), manufactured by Kawaken Fine Chemicals Co., Ltd., trade name: “ASBD” was used.

### Titanium Alkoxide (B1): Comparative Example 1

In Comparative Example 1, titanium alkoxide (B1) was used instead of aluminum alkoxide (B). As the titanium alkoxide (B1), trade name: “Orgatics TA-25” manufactured by Matsumoto Fine Chemical Co., Ltd. was used.

### Zirconium Alkoxide (B2): Comparative Example 2

In Comparative Example 2, zirconium alkoxide (B2) was used instead of aluminum alkoxide (B). As the zirconium alkoxide (B2), trade name: “Orgatics ZA-65” manufactured by Matsumoto Fine Chemical Co., Ltd. was used.

### Silicon Alkoxide (C): Examples 1 to 6, Comparative Examples 1 and 2

As the silicon alkoxide (C), KC-89S (manufactured by Shin-Etsu Chemical Co., Ltd.) as silicon alkoxide oligomer was used.

### <Evaluation Method>

### (Curability)

Each of the above materials was prepared by a blending amount shown in Table 1, and a predetermined amount was put into a mold made of a fluororesin, and then, after heating at 70° C. for 24 hours, at 105° C. for 24 hours, and 48 hours at 150° C., respectively, a 50 mm×50 mm×5 mm elastic member was prototyped. Each prototype elastic member was tested by tactual sense, and the presence or absence of “stickiness” was evaluated. If the stickiness is large, as shown in FIG. 10, there is a possibility that when brought into contact with and pressed against the skin outer surface 51, the elastic member is not deformed along the skin outer surface 51 but projects to the outer side of the frame-shaped member 13b. In Table 1, the elastic members without stickiness are indicated by “A”, and the elastic members having stickiness are indicated. by “B”.

### (Ultraviolet Light Transmittance)

A 1 mm thick cured product obtained by curing each of the above materials, prepared by the blending amount shown in Table 1, in a Teflon (registered trademark) Petri dish was used as a measurement target. The curing method is the same as described above. Then, ultraviolet light having a wavelength of 280 nm to 315 nm was applied while being changed in steps of 1 nm, and whether or not the transmittance at all wavelengths was 90% or more was evaluated. At all wavelengths, the cured products having a transmittance of 90% or more are indicated by “A”, and the others are indicated by “B”.

### (Elasticity)

A 50 mm×50 mm×5 mm elastic member was prototyped in the same manner as in the evaluation of the curability, and this elastic member was placed on a side surface of a cylindrical vinyl chloride pipe having a diameter of 200 mm. Then, when a 1 kg iron plate was placed on the elastic member, it was evaluated whether or not the entire 50 mm×50 mm surface of the elastic member was in contact with the side surface (curved surface) of the cylinder. The elastic members whose entire surface is in contact are indicated by “A”, and the others are indicated by “B”.

### (Bending Strength)

A 1 mm thick cured product obtained by being cured in a Teflon (registered trademark) petri dish was used as a measurement target in the same manner as in the evaluation of the ultraviolet light transmittance, When the cured product was bent by hand, it was evaluated whether the cured product was broken. The cured products that were not broken when bent are indicated by “A”, and the broken cured products are indicated by “B”.

### (Comprehensive Evaluation)

In all items including curability, ultraviolet light transmittance, elasticity, and bending strength, the case where the evaluation of “A” was obtained was judged as “A”, and the case where the evaluation of “B” was obtained even in some items was judged as “B”. Comparative Examples 1 and 2 were not evaluated in the elasticity and bending strength tests because transmittance for ultraviolet light was low. Table 1 below shows that when the elastic member 11 is formed of each of the compositions of Examples 1 to 6, both high transmittance of 90% or more for ultraviolet light and bending characteristics (elasticity) are realized simultaneously.

TABLE 1

| | | | | | | | | | Comparative | Comparative |
|---|---|---|---|---|---|---|---|---|---|---|
| | | | Example 1 | Example 2 | Example 3 | Example 4 | Example 5 | Example 6 | Example 1 | Example 2 |
| Material | Dimethyl- | Low molecular | 5.0 | 5.0 | 0.0 | 0.0 | 50.0 | 50.0 | 5.0 | 5.0 |
| | polysiloxane (A) | weight (A1) | | | | | | | | |
| | | High molecular | 45 | 45 | 50 | 50 | 0 | 0 | 45 | 45 |
| | | weight (A2) | | | | | | | | |

| | | | | | | | | | |
|---|---|---|---|---|---|---|---|---|---|
| | Aluminum alkoxide (B) | 0.02 | 0.045 | 0.02 | 0.15 | 0.02 | 0.15 | 0 | 0 |
| | Titanium alkoxide (B1) | 0 | 0 | 0 | 0 | 0 | 0 | 0.15 | 0 |
| | Zirconium alkoxide (B2) | 0 | 0 | 0 | 0 | 0 | 0 | 0 | 0.15 |
| | Silicon alkoxide (C) | 1.0 | 1.0 | 1.0 | 1.0 | 1.0 | 1.0 | 1.0 | 1.0 |
| Evaluation | Curability | A | A | A | A | A | A | A | A |
| | Ultraviolet light transmittance | A | A | A | A | A | A | B | B |
| | Elasticity | A | A | A | A | A | A | — | — |
| | Bending strength | A | A | A | A | A | A | — | — |
| | Comprehensive evaluation | A | A | A | A | A | A | B | B |

### [Verification]

The fact that the irradiance of ultraviolet light to an affected area is improved by the ultraviolet irradiation device 1 will be verified below,

### <Thickness of Elastic Member 11>

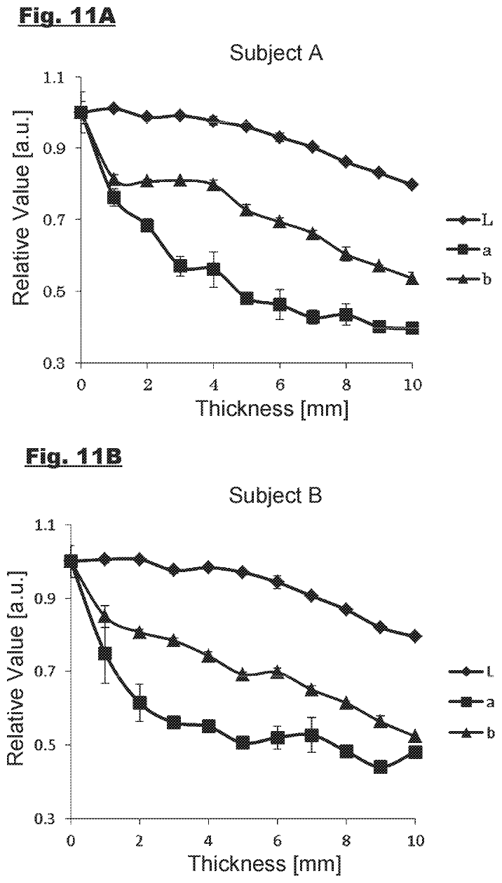

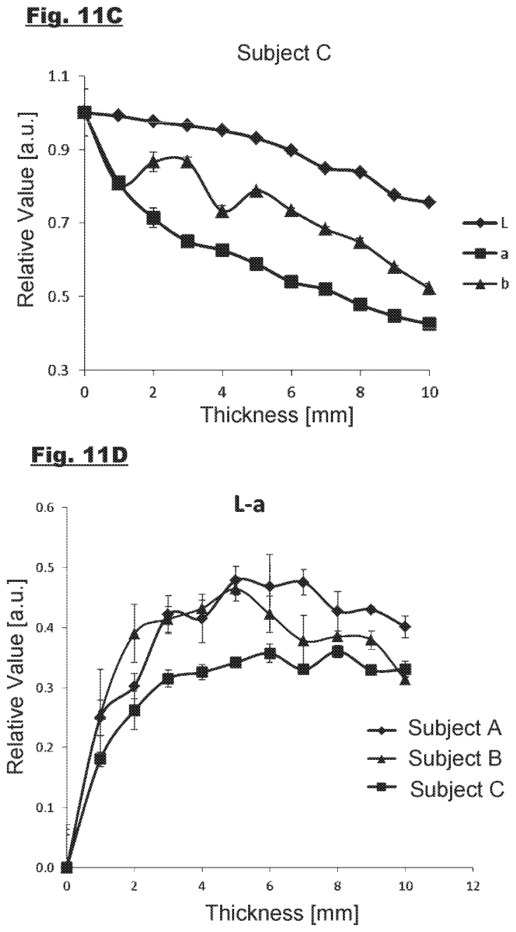

Hemoglobin has heme, which is a red pigment, and is reddish. FIGS. 11A to 11C are graphs showing results obtained when the elastic members 11 having different thicknesses were pressed with a pressure of 12 kPa against the skins of three subjects (A, B, C), a color difference of the skin was measured by the SCI (Specular Component Include) method.

The numerical value of 12 kPa is, for example, a value calculated based on each value, assuming that an area of a region of the elastic member 11 in contact with the skin outer surface 51 is 855 mm², the weight of the device body 3 is 670 g, and the load f1 is 1 kgf (≈10 N) in order to apply a slight load to the irradiation subject 50 in a state where the device body 3 is gripped.

In each drawing, the horizontal axis indicates the thickness of the elastic member 11. A thickness of 0 mm on the horizontal axis corresponds to a state without the elastic member 11 (initial state). In each drawing, the vertical axis represents values of L, a, and b, and is shown as a relative value when a value at a thickness of 0 mm is 1.

Among the values measured by the SCI method, the a value is a value indicating a degree of redness, and it is considered that hemoglobin is retracted as the a value decreases. The L value is a value indicating lightness, and as the L value decreases, the color approaches black and absorbs light. Thus, by reducing the a value within a range that does not cause the decrease in the L value, absorption by the hemoglobin or the like until incident ultraviolet light reaches an affected area is suppressed.

FIG. 11D is a graph showing results obtained by calculating a difference value (L−a) between the L value and the a value for each thickness of the elastic member 11 for each of the subjects A, B, and C. According to FIG. 11D, the graph shows a maximum value when the thickness is in a range of 5 mm or more and 8 mm or less. Therefore, it can be seen that when the thickness is in the range of 5 mm or more and 8 mm or less, the a value can be reduced while suppressing the decrease in the L value. The effect of the present invention is sufficiently exhibited when the thickness of the elastic member 11 is in the range of 3 mm or more and 10 mm or less.

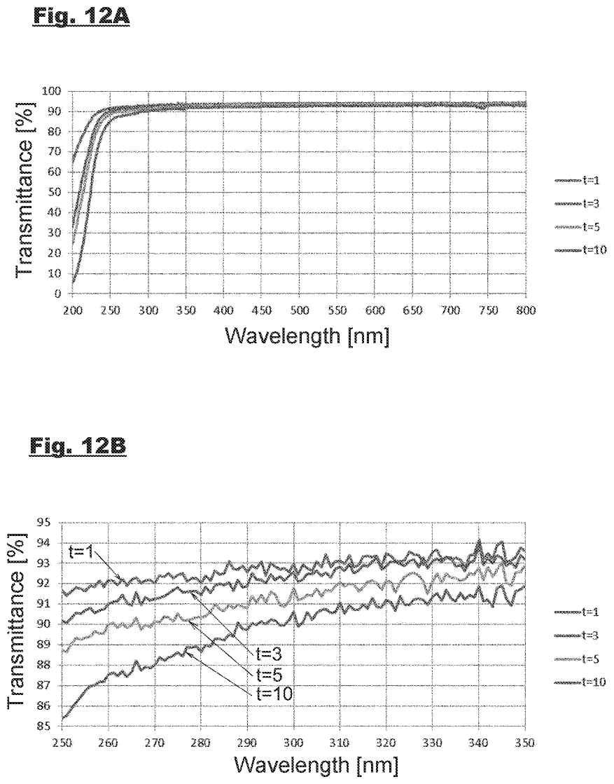

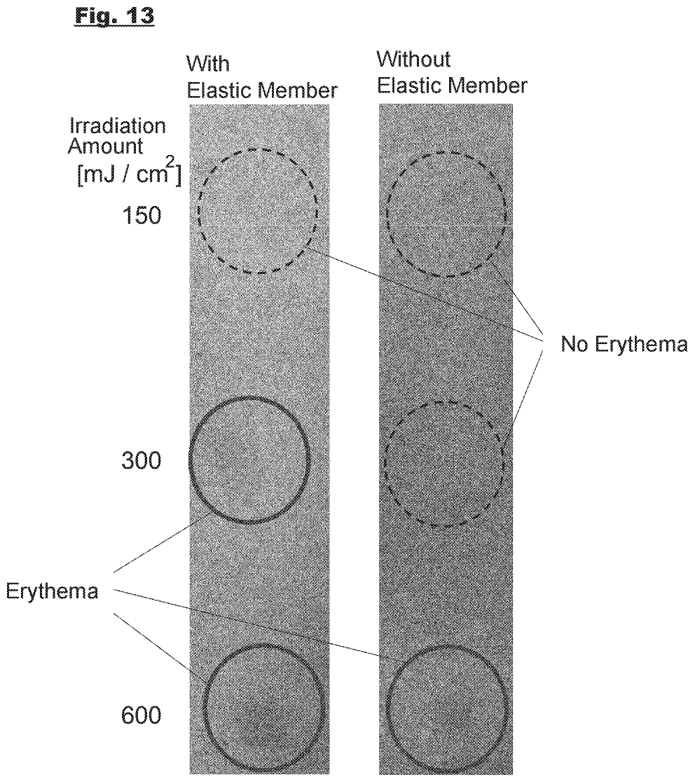

FIGS. 12A and 12B are graphs showing results obtained by measuring a transmittance spectrum with respect to light in a state where a thickness t of the elastic member 11 is changed to 1 mm, 3 mm, 5 mm, and 10 mm. Here, as the material of the elastic member 11, a material prepared under the conditions of Example 1 is used. FIG. 12B is a graph obtained by enlarging a partial wavelength region of FIG. 12A. In each graph, the horizontal axis indicates the wavelength, and the vertical axis indicates the transmittance.

FIGS. 12A and 12B show that the transmittance to light is improved as the thickness of the elastic member 11 decreases. It is confirmed that 90% or more of UVB (wavelength: 320 nm to 400 nm) light is transmitted when the thickness is in a range of 10 mm or less. It is confirmed that even within the wavelength range of 290 nm to 320 nm, if the thickness is 5 mm or less, 90% or more transmittance is provided, and even if the thickness is more than 5 mm and 10 mm or less, almost 90% or more transmittance is exhibited.

### <Measurement of Minimal Erythema Dose (MED)>

A comparison was made between the case where a subject D (irradiation subject 50) was irradiated with the ultraviolet light L1 via the elastic member 11 and the case where the subject D was irradiated with the ultraviolet light L1 without the elastic member 11. The elastic member 11 had a size of 50 mm×50 mm×5 mm, and an irradiation area was 10 mm square. At the time of irradiation, an aluminum foil was wound three times around an upper surface of the skin outer surface 51, and a hole was made in a portion corresponding to the irradiation region to form a mask.

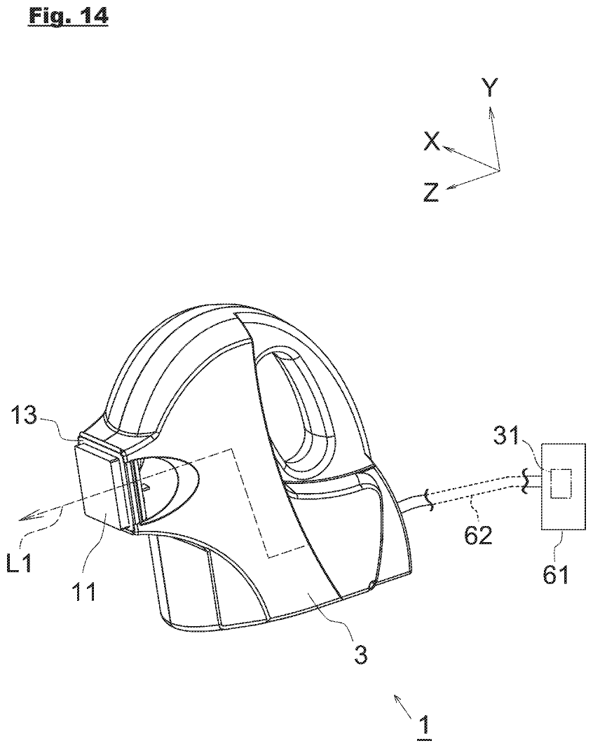

A peak wavelength of the light source 31 was 308 nm, and while an irradiation amount was changed to 150, 300, and 600 mK/cm², the irradiation amount at which the irradiation region of the skin outer surface 51 became red for the first time was specified to measure the minimal erythema dose (MED). The results are shown in the photograph of FIG. 13.

According to the results shown in FIG. 13, it is confirmed that the MED is reduced when the elastic member 11 is provided and the ultraviolet light L1 is applied, compared with the case where the ultraviolet light L1 is applied without the elastic member 11. Thereby, it was confirmed that the amount (irradiance) of the ultraviolet light reaching the skin (inside) was increased by applying the ultraviolet light L1 through the elastic member 11.

### [Another Embodiment]

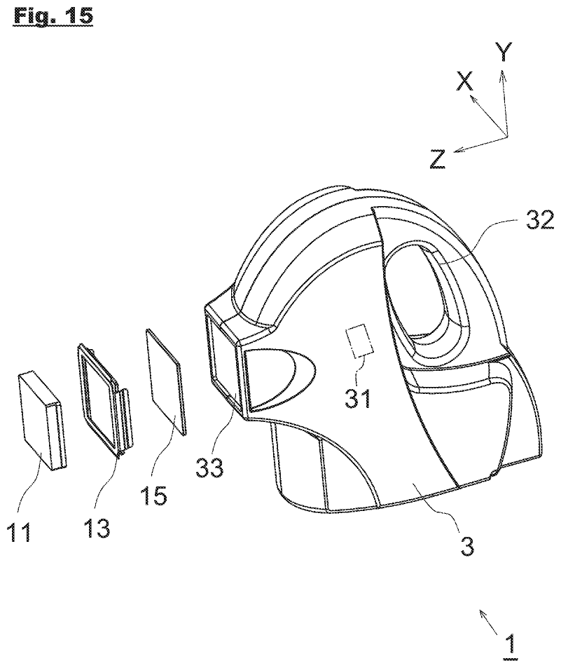

Hereinafter, another embodiment of the ultraviolet irradiation device 1 will be described.

<1> In the embodiment described above, the case where the device body 3 incorporates the light source 31 has been described, but the light source 31 may be disposed outside the device body 3. For example, as shown in FIG. 14, a light source device 61 different from the device body 3 is provided, and ultraviolet light emitted from the light source 31 built in the light source device 61 may be guided to the device body 3 via a light guide member 62. As the light guide member 62, for example, an optical fiber or the like can be used.

<2> In the example illustrated in FIG. 4, the substrate 15 is illustrated as being built in the device body 3, but as illustrated in FIG. 15, the substrate 15 may be removable from the device body 3.

<3> The shapes of the device body 3, the elastic member 11, and the attachment 13 described above are merely examples, and various modifications are possible within the scope of achieving the object of the present invention.

### DESCRIPTION OF REFERENCE SIGNS

1 Ultraviolet irradiation device

3 Device body

11 Elastic member

11a First surface of elastic member

11b Second surface of elastic member

11c Step portion of elastic member

13 Attachment

13a Opening region

13b Frame-shaped member

13c Claw portion

15 Substrate

31 Light source

32 Gripping portion

33 Light emitting portion

41 Operator

50 Irradiation subject

51 Skin outer surface

61 Light source device

62 Light guide member

L1 Ultraviolet light

1. An ultraviolet irradiation device comprising:
a device body configured to emit ultraviolet light from a light emitting portion;
a substrate disposed at the light emitting portion, exhibiting transparency to the ultraviolet light, and including a first surface and a second surface facing the first surface; and
an elastic member placed on the second surface of the substrate opposite to the first surface located on the device body side and formed of a material having transparency to the ultraviolet light.

2. The ultraviolet irradiation device according to claim 1, further comprising an attachment formed of a frame-shaped member including an opening region and detachably attached to the device body,
wherein the elastic member is fitted into the opening region, and an outer peripheral portion of the elastic member is fixed to the device body via the attachment.

3. The ultraviolet irradiation device according to claim 2, wherein the elastic member has a first surface located on a side closer to the substrate and a second surface opposite to the first surface, and the second surface is disposed projecting opposite to the device body relative to the attachment.

4. The ultraviolet irradiation device according to claim 3, wherein the elastic member has a step portion formed at a position between the first surface and the second surface, and
when the frame-shaped member of the attachment comes into contact with the step portion, the elastic member is fitted into the opening region.

5. The ultraviolet irradiation device according to claim 1, wherein the elastic member has a thickness of 3 mm to 10 mm.

6. The ultraviolet irradiation device according to claim 1, wherein the device body includes an ultraviolet light source.

7. The ultraviolet irradiation device according to claim 1, wherein the elastic member comprises an organic-inorganic hybrid composition (X), and
the organic-inorganic hybrid composition (X) has no phenyl group in its molecule, has only a methyl group in its side chain, and has a skeleton composed of dimethylpolysiloxane having a hydroxy terminal.

8. The ultraviolet irradiation device according to claim 7, wherein the organic-inorganic hybrid composition (X) is a product formed by dehydration-condensation of dimethylpolysiloxane (A), aluminum alkoxide (B), and silicon alkoxide (C).

9. An ultraviolet irradiation method comprising:
placing an elastic member, formed of a material having transparency to ultraviolet light, on a surface of an irradiation target region; and
in a state where in a surface of the elastic member, which is opposite to the irradiation target region, in a substrate exhibiting transparency to ultraviolet light and including a first surface and a second surface facing the first surface, the second surface is in contact with the elastic member, applying the ultraviolet light to the first surface and the second surface of the substrate and the irradiation target region via the elastic member.

10. An attachment for use in the ultraviolet irradiation device according to claim 2.

11. An elastic member for use in the ultraviolet irradiation device according to claim 7.
